(19) United States
(12) Patent Application Publication
(10) Pub. No.: US 2023/0404379 A1
(43) Pub. Date: Dec. 21, 2023

(54) ENDOSCOPE

(71) Applicant: FUJIFILM Corporation, Tokyo (JP)

(72) Inventor: Masaya INOUE, Kanagawa (JP)

(73) Assignee: FUJIFILM Corporation, Tokyo (JP)

(21) Appl. No.: 18/459,438

(22) Filed: Sep. 1, 2023

Related U.S. Application Data
(63) Continuation of application No. PCT/JP2022/002875, filed on Jan. 26, 2022.

(30) Foreign Application Priority Data
Mar. 3, 2021 (JP) 2021-033546

(51) Int. Cl. A61B 1/018; A61B 1/005

(52) U.S. Cl. CPC A61B 1/018; A61B 1/0052; A61B 1/0055

(57) ABSTRACT

An endoscope according to the embodiment of the present invention includes an elevator operation lever that displaces an elevation angle of a treatment tool elevator for elevating a treatment tool provided in a distal end of an insertion part, the elevator operation lever including a coupling part coupled to a rotational movement member that moves rotationally about the same shaft as a rotational movement shaft of an angle knob, and being coupled to the rotational movement member as an engaging member is provided in any one of the rotational movement member or the coupling part and engages with an engaging hole provided in the other one of the rotational movement member or the coupling part.

### CROSS-REFERENCE TO RELATED APPLICATIONS

This application is a Continuation of PCT International Application No. PCT/JP2022/002875 filed on 26 Jan. 2022, which claims priority under 35 U.S.C § 119(a) to Japanese Patent Application No. 2021-033546 filed on 3 Mar. 2021. The above application is hereby expressly incorporated by reference, in its entirety, into the present application.

### BACKGROUND OF THE INVENTION

### 1. Field of the Invention

The present invention relates to an endoscope comprising an elevator operation lever that is easily attachable and detachable.

### 2. Description of the Related Art

In an endoscope into which a treatment tool can be inserted, there is known an endoscope in which an elevator provided in a distal end of an insertion part and an elevator operation lever of an operating part are coupled to each other, and a posture of the elevator is displaced by rotationally moving and operating the elevator operation lever, thereby changing a protruding direction of the treatment tool. Conventionally, an elevator operation lever or a bending operating member that bends an insertion part is, as disclosed in JP2015-104424A (corresponding to US2015/0148598A1) for example, fixed to an operating part by a method that needs a tool that is tightened with a screw.

### SUMMARY OF THE INVENTION

However, in recent years, since an endoscope in which a bending operating member attached to an operating part of the endoscope is easily attachable and which is easily assembled and disassembled has become widespread, even in an elevator operation lever, a method by which an elevator operation lever is easily attachable and detachable is desired.

An object of the present invention is to provide an endoscope comprising an elevator operation lever that is easily attachable and detachable.

The present invention has been made to solve the problem described above, and an endoscope according to an aspect of the present invention comprises a plurality of angle knobs that perform a bending operation of an insertion part, a lock lever that locks or unlocks rotational movement of the angle knob disposed in a body part side of an operating part among the plurality of angle knobs; and an elevator operation lever that displaces an elevation angle of a treatment tool elevator for elevating a treatment tool provided in a distal end of the insertion part, the elevator operation lever including a coupling part coupled to a rotational movement member that moves rotationally about the same shaft as a rotational movement shaft of the angle knob, and being coupled to the rotational movement member as an engaging member is provided in any one of the rotational movement member or the coupling part and engages with an engaging hole provided in the other one of the rotational movement member or the coupling part.

It is preferable that the elevator operation lever is supported between the lock lever and the rotational movement member in a state where the lock lever is attached and is attachable and detachable by taking off the lock lever.

It is preferable that the engaging member is provided in the rotational movement member, and the engaging hole is provided in the coupling part.

It is preferable that the lock lever is disposed in the body part side of the operating part from the plurality of angle knobs.

It is preferable that the plurality of angle knobs are an up-down angle knob that bends the insertion part in an up-down direction and a right-left angle knob that bends the insertion part in a right-left direction, the up-down angle knob is disposed in the body part side of the operating part from the right-left angle knob, and the lock lever is an up-down lock lever that locks or unlocks rotational movement of the up-down angle knob.

It is preferable that the up-down angle knob, the right-left angle knob, and the up-down lock lever are attached attachably and detachably with respect to the operating part.

According to an aspect of the present invention, it is possible to provide an endoscope comprising the elevator operation lever that is easily attachable and detachable.

### BRIEF DESCRIPTION OF THE DRAWINGS

FIG. 1 is an overall view of an endoscope according to an aspect of the present invention.

FIG. 2 is an enlarged plan view of an operating part of the endoscope.

FIG. 3 is a perspective view describing a rotational movement range of a treatment tool protruding from a distal end.

FIG. 4 is an exploded view describing an elevator rotational movement mechanism.

FIG. 5 is an exploded view describing an operating member attached to the operating part of the endoscope.

FIG. 6 is a perspective view describing a state where an elevator operation lever is attached.

FIG. 7 is a partial cross-sectional view in a state where an elevator operation lever is attached.

### DESCRIPTION OF THE PREFERRED EMBODIMENTS

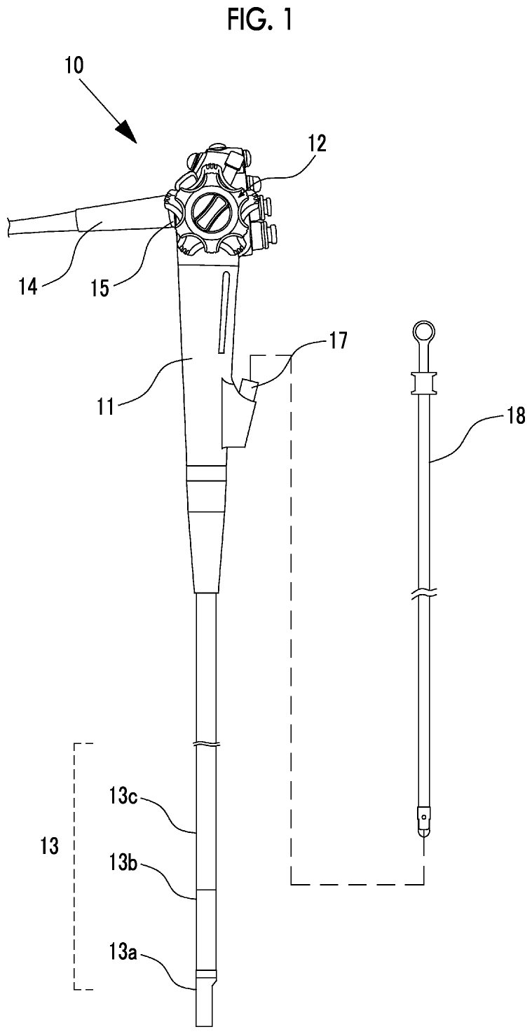

As shown in FIG. 1, an endoscope 10 has a gripped part 11 gripped by an operator, an operating part 12 for performing various operations, an insertion part 13 inserted into a subject, and a universal cord 14 connected to a connecting part 15. An illumination device (not shown) that supplies illumination light to the endoscope 10 and a processor device (not shown) that executes various kinds of processing according to an operation of the operating part 12 are connected to the endoscope 10 via the universal cord 14. In the insertion part 13, a distal end 13a, a bendable part 13b, and a flexible part 13c are coupled in this order from the distal end side toward the proximal end side.

The endoscope 10 is, for example, a side-view endoscope such as a duodenoscope, and is provided with a treatment tool insertion port 17 into which a treatment tool 18 used for performing a medical treatment such as a therapy or an examination is inserted. The treatment tool 18 that is inserted passes through an insertion path disposed inside the insertion part 13 from the treatment tool insertion port 17 to the distal end 13a, protrudes from the distal end 13a, and is used for various medical treatments.

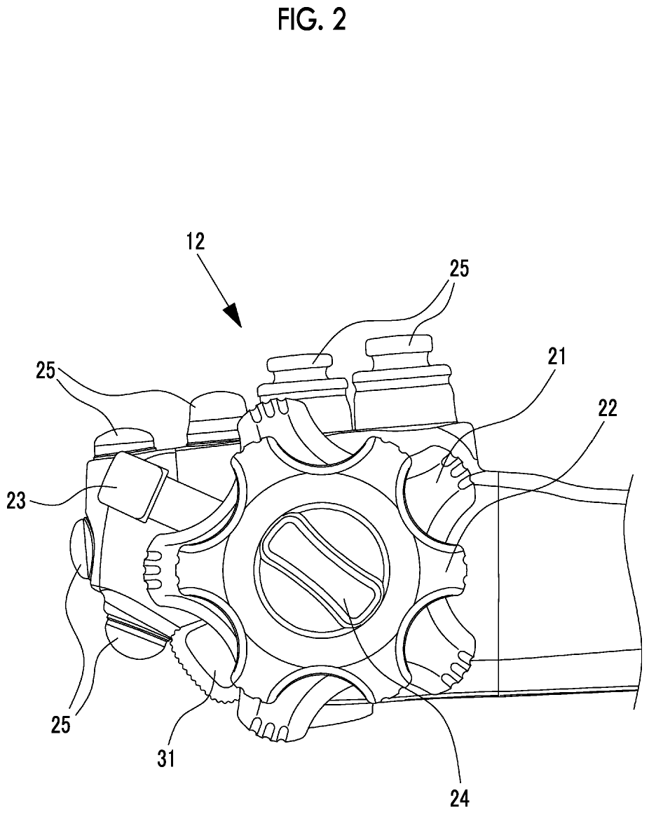

As shown in FIG. 2, the operating part 12 has, as bending operating members, an up-down angle knob 21 that bends the bendable part 13b in an up-down direction, a right-left angle knob 22 that bends the bendable part 13b in a right-left direction, and an up-down lock lever 23 and a right-left lock handle 24 that locks or unlocks rotational movement of an angle knob by being rotationally moved and operated. By rotationally moving and operating the up-down and right-left angle knobs, the bendable part 13b bends and the distal end 13a is directed to a desired direction. In order to maintain a bent state of the bendable part 13b, the up-down lock lever 23 and the right-left lock handle 24 are appropriately rotationally moved and operated. An outer peripheral surface of a body part 41 of the operating part 12 is provided with a plurality of operation buttons 25, and, by pushing down an operation button 25, the endoscope 10 and the processor device cooperate with each other to execute various kinds of processing.

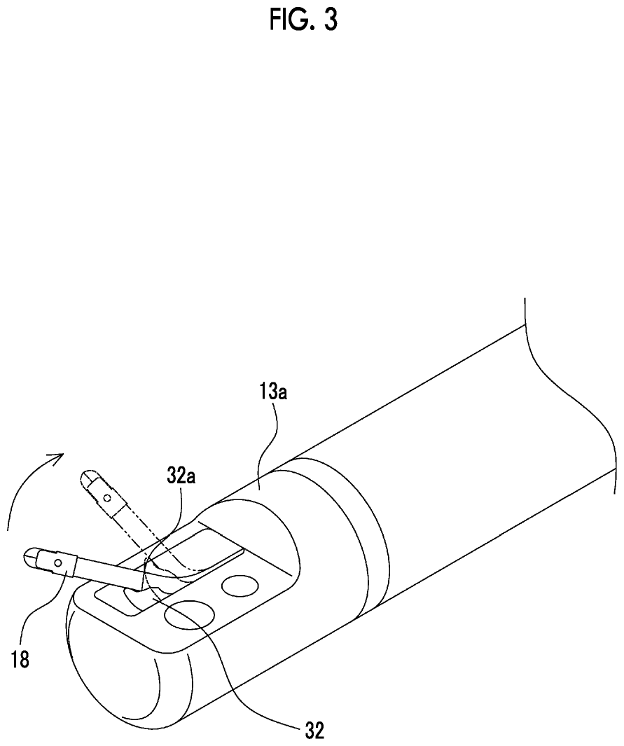

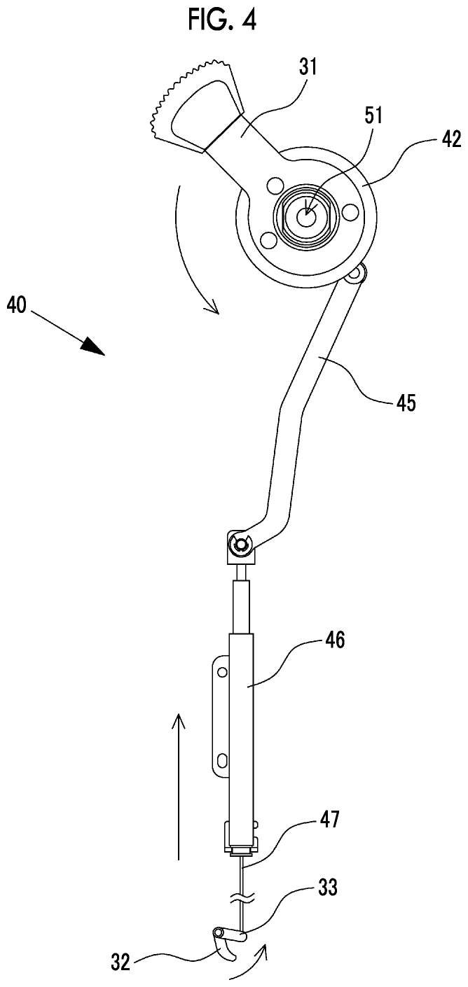

In addition, the operating part 12 is provided with an elevator operation lever 31. The elevator operation lever 31 is connected to a treatment tool elevator 32 (hereinafter, simply referred to as an elevator) provided in the distal end 13a shown in FIG. 3 by a pulling wire 47 (refer to FIG. 4) inserted into the endoscope 10, and the elevator 32 is changed from a lowered posture to an elevated posture by an elevator rotational movement mechanism 40 that will be described later by rotationally moving the elevator operation lever 31. In a case of performing various medical treatments, the treatment tool 18 protrudes from the distal end 13a along a guide surface 32a of the elevator 32, and a protruding direction changes in accordance with a change in the posture of the elevator 32.

FIG. 4 is a schematic view describing the elevator rotational movement mechanism 40. The elevator rotational movement mechanism 40 has a rotational movement member 42 that moves rotationally about a rotational movement shaft 51, a crank member 45 coupled to the rotational movement member 42, and a slider 46 coupled to the crank member 45. The slider 46 is connected to an elevator rotational movement lever 33 for rotationally moving the elevator 32 by the pulling wire 47. The crank member 45 is a member that converts a rotational motion of the rotational movement member 42 into a linear motion of the slider 46. In addition, the elevator rotational movement lever 33 is coupled such that an angle of the elevator 32 changes in a case where the elevator rotational movement lever 33 moves rotationally. Accordingly, in a state where the elevator operation lever 31 is coupled to the rotational movement member 42, by rotationally moving and operating the elevator operation lever 31, the pulling wire 47 connected to the rotational movement member 42 is pushed and pulled, and the posture of the elevator 32 changes with the rotational movement of the elevator rotational movement lever 33. For example, as shown in FIG. 4, in a case where the elevator operation lever 31 is rotationally moved in a left rotation direction, the pulling wire 47 is pulled, and the elevator 32 moves rotationally in an elevated posture direction. In the elevator rotational movement mechanism 40, the elevator rotational movement lever 33 may be omitted, and the pulling wire 47 and the elevator 32 may be connected directly.

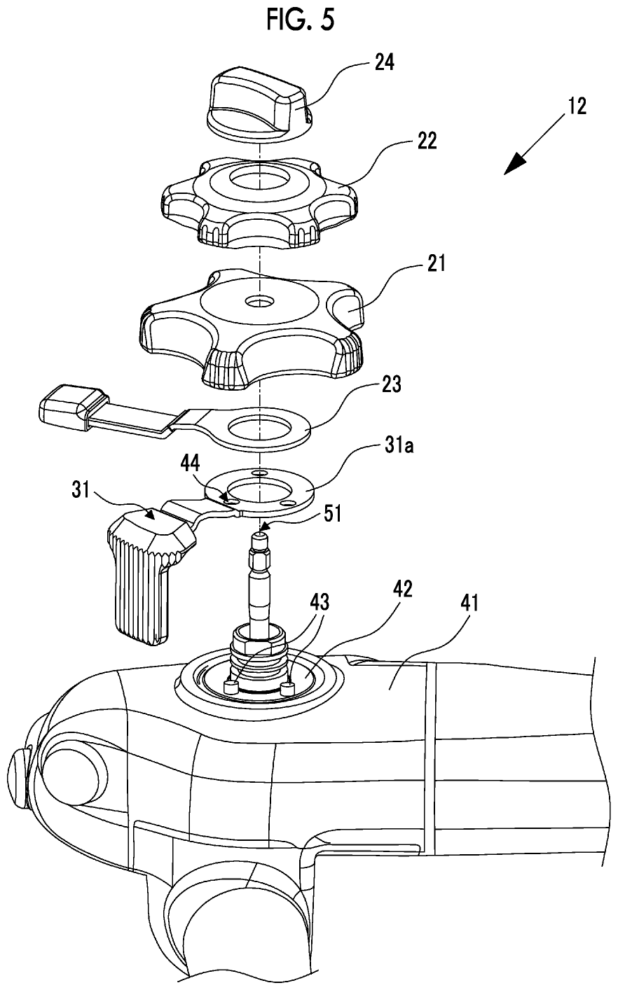

As shown in FIG. 5, the respective bending operating members and the elevator operation lever 31 are attached to the body part 41 of the operating part 12 such that the right-left lock handle 24, the right-left angle knob 22, the up-down angle knob 21, the up-down lock lever 23, and the elevator operation lever 31 are stacked in this order from an outside with respect to the same rotational movement shaft 51. In a case of attachment, the elevator operation lever 31 is first attached to the rotational movement shaft 51, and then the up-down lock lever 23, the up-down angle knob 21, the right-left angle knob 22, and the right-left lock handle 24 are attached to the rotational movement shaft 51 in this order such that attachment portions (not shown) provided in a center of the respective bending operating members and the elevator operation lever 31 are put to the rotational movement shaft 51. In a case where the members are attached to the body part 41, the members move rotationally independently of each other. In the rotational movement member 42 to which the elevator operation lever 31 is attached, a plurality of an engaging member 43 is provided so as to be fixed to the rotational movement member 42. An engaging hole 44 that engages with the engaging member 43 is formed in an annular operation lever coupling part (coupling part of the embodiment of the present invention) 31a of the elevator operation lever 31, and the engaging member 43 and the engaging hole 44 engage with each other, thereby attaching the elevator operation lever 31 to the rotational movement member 42.

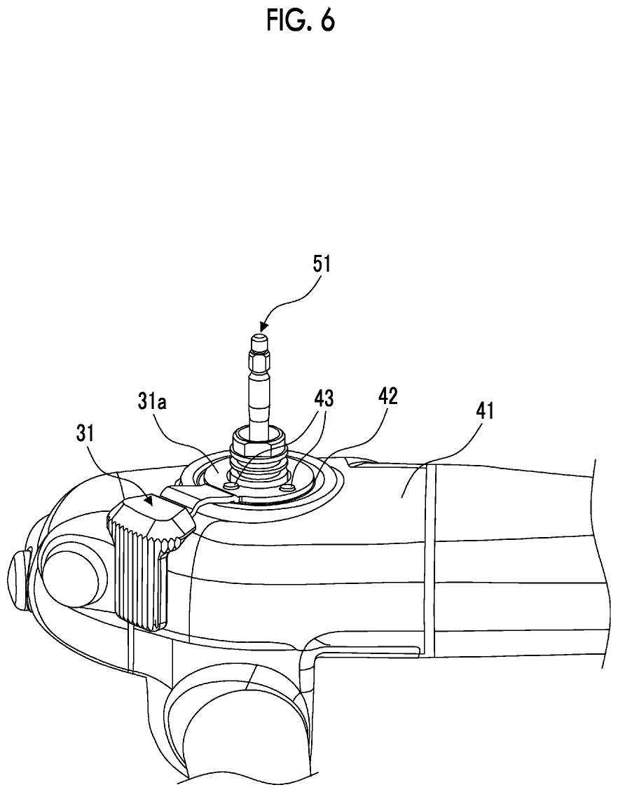

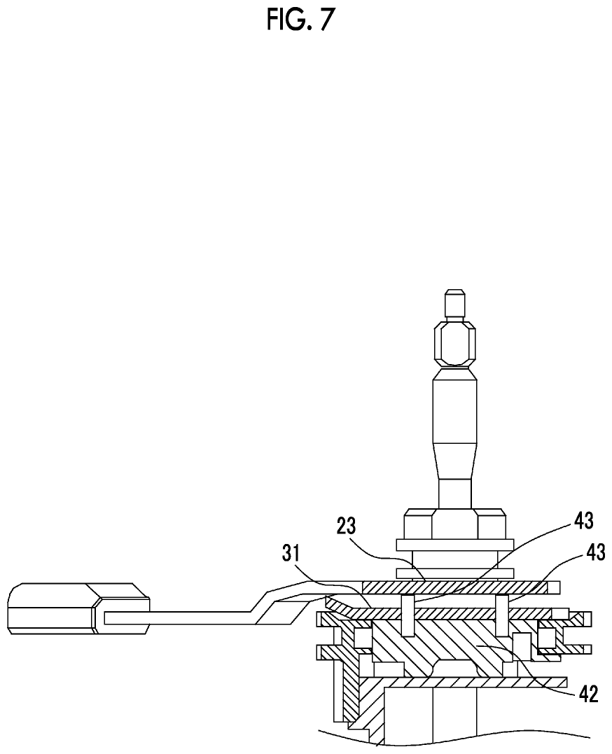

As shown in FIG. 6, the engaging member 43 is provided so as to protrude from an upper surface of the operation lever coupling part 31a in a state where the elevator operation lever 31 is attached to the rotational movement member 42. FIG. 7 is a schematic cross-sectional view in a state where the elevator operation lever 31 is attached to the rotational movement member 42. In a state where only the elevator operation lever 31 is attached (refer to FIG. 6), the elevator operation lever 31 is not fixed with respect to a direction perpendicular to the rotational movement member 42 (direction of the rotational movement shaft 51). However, in a state where each of the bending operating members is attached, since the up-down lock lever 23 is disposed in an outside of the elevator operation lever 31, the elevator operation lever 31 is supported between the rotational movement member 42 and the up-down lock lever 23.

As described above, in a state where each of the bending operating members and the elevator operation lever 31 are attached, the elevator operation lever 31 is supported between the up-down lock lever 23 and the rotational movement member 42, and the elevator 32 can be rotationally moved and operated. In addition, in a case where the operating part 12 is disassembled or assembled, or the elevator operation lever 31 is replaced, since the elevator operation lever 31 is not fixed by a member that needs a tool such as a screw, the elevator operation lever 31 can be easily attached and detached by taking off each of the bending operating members.

In FIGS. 5 and 6, a plurality of columnar members are provided as the engaging member 43, the engaging member 43 may have a prismatic shape or have other shapes. In this case, it is preferable to provide the engaging hole 44 in accordance with the shape of the engaging member 43. The number of engaging members 43 may be single, and the operation lever coupling part 31a and the rotational movement member 42 may be configured to engage with and be coupled to each other by the rotational movement shaft 51 and the single engaging member 43.

In the embodiment described above, the engaging member 43 is provided in the rotational movement member 42 and the engaging hole 44 is provided in the operation lever coupling part 31a, but the engaging member 43 and the engaging hole 44 may be provided in switched positions. That is, the rotational movement member 42 may be provided with the engaging hole 44, and the operation lever coupling part 31a may be provided with the engaging member 43.

### EXPLANATION OF REFERENCES

10: endoscope
11: gripped part
12: operating part
13: insertion part
13a: distal end
13b: bendable part
13c: flexible part
14: universal cord
15: connecting part
17: treatment tool insertion port
18: treatment tool
21: up-down angle knob
22: right-left angle knob
23: up-down lock lever
24: right-left lock handle
25: operation button
31: elevator operation lever
31a: operation lever coupling part
32: treatment tool elevator (elevator)
32a: guide surface
33: elevator rotational movement lever
40: elevator rotational movement mechanism
41: body part
42: rotational movement member
43: engaging member
44: engaging hole
45: crank member
46: slider
47: pulling wire
51: rotational movement shaft

What is claimed is:

1. An endo scope comprising:
a plurality of angle knobs that are operated to perform a bending operation of an insertion part;
a lock lever that is operated to lock or unlock rotational movement of the angle knob disposed in a body part side of an operating part among the plurality of angle knobs; and
an elevator operation lever that is operated to displace an elevation angle of a treatment tool elevator for elevating a treatment tool provided in a distal end of the insertion part,
wherein the elevator operation lever includes a coupling part coupled to a rotational movement member that moves rotationally about the same shaft as a rotational movement shaft of the angle knob,
an engaging member is provided in any one of the rotational movement member or the coupling part, and
the elevator operation lever is coupled to the rotational movement member by engaging the engaging member with an engaging hole provided in the other one of the rotational movement member or the coupling part.

2. The endoscope according to claim 1,
wherein the elevator operation lever is supported between the lock lever and the rotational movement member in a state where the lock lever is attached, and is attachable and detachable by taking off the lock lever.

3. The endoscope according to claim 1,
wherein the engaging member is provided in the rotational movement member, and the engaging hole is provided in the coupling part.

4. The endoscope according to claim 1,
wherein the lock lever is disposed in the body part side of the operating part from the plurality of angle knobs.

5. The endoscope according to claim 1,
wherein the plurality of angle knobs are an up-down angle knob that is operated to bend the insertion part in an up-down direction and a right-left angle knob that is operated to bend the insertion part in a right-left direction,
the up-down angle knob is disposed in the body part side of the operating part from the right-left angle knob, and
the lock lever is an up-down lock lever that is operated to lock or unlock rotational movement of the up-down angle knob.

6. The endoscope according to claim 5,
wherein the up-down angle knob, the right-left angle knob, and the up-down lock lever are attached attachably and detachably with respect to the operating part.
